(19) United States
(12) Patent Application Publication
(10) Pub. No.: US 2022/0040274 A1
(43) Pub. Date: Feb. 10, 2022

(54) METHOD FOR BLOCKING ANTIBODY ADMINISTRATION IN TRANSPLANTATION

(71) Applicant: National Institute of Transplantation Foundation, Los Angeles, CA (US)

(72) Inventors: James Cicciarelli, Los Angeles, CA (US); Nori Kasahara, Los Angeles, CA (US); Nathan Lemp, Los Angeles, CA (US)

(21) Appl. No.: 17/327,733

(22) Filed: May 23, 2021

Related U.S. Application Data
(63) Continuation of application No. 14/134,342, filed on Dec. 19, 2013.
(60) Provisional application No. 61/739,603, filed on Dec. 19, 2012.

(51) Int. Cl. A61K 39/00; C07K 16/28; C07K 16/00

(52) U.S. Cl. CPC A61K 39/0005; C07K 2317/52; C07K 16/00; C07K 16/2833

(57) ABSTRACT

A method can include determining a donor-specific human leukocyte antigen of a tissue transplant recipient; determining a ratio of the tissue transplant recipient for a level of an immunoglobulin G subclass 4 antibody (G4) for the antigen with respect to a level of another immunoglobulin G subclass antibody for the antigen; and, based on the ratio, deciding to administer an amount of the G4 to the tissue transplant recipient.

### RELATED APPLICATION

This application is a continuation of a pending US Patent Application having application Ser. No. 14/134,342, filed 19 Dec. 2013, which is incorporated by reference herein and which claims priority to and the benefit of a U.S. Provisional Patent Application No. 61/739,603, filed on 19 Dec. 2012 (expired), which is incorporated by reference herein.

### TECHNICAL FIELD

Subject matter disclosed herein relates generally to immunological technologies and techniques.

### BRIEF DESCRIPTION OF THE DRAWINGS

A more complete understanding of the various methods, devices, assemblies, systems, arrangements, etc., described herein, and equivalents thereof, may be had by reference to the following detailed description when taken in conjunction with examples shown in the accompanying drawings where:

FIG. 1 is a table (Table 1);

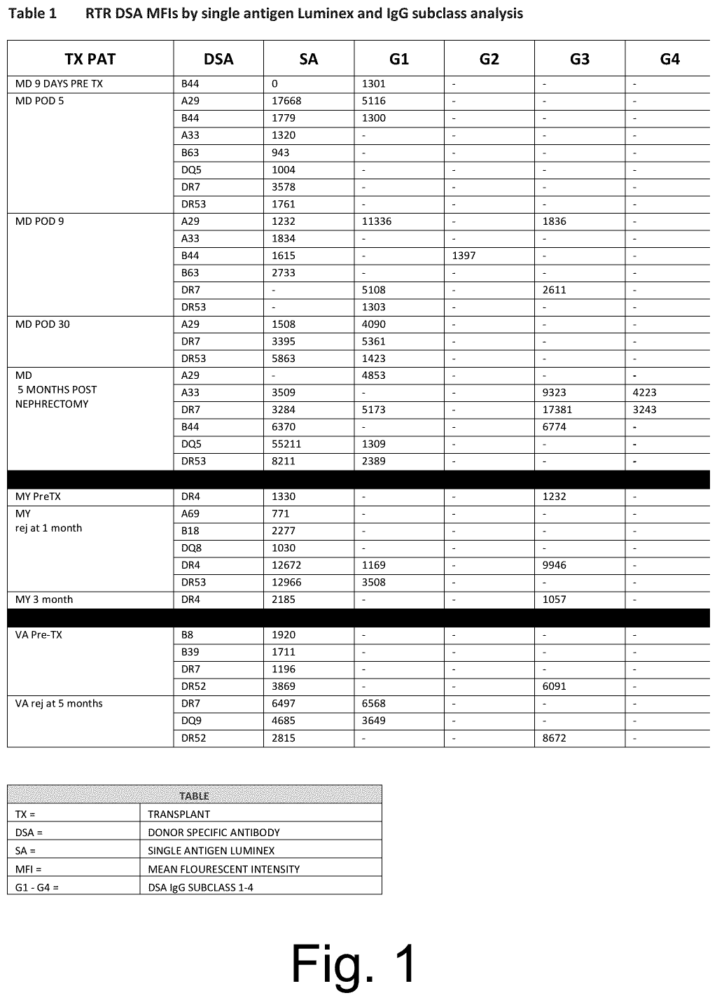

FIG. 2 is a table (Table 2);

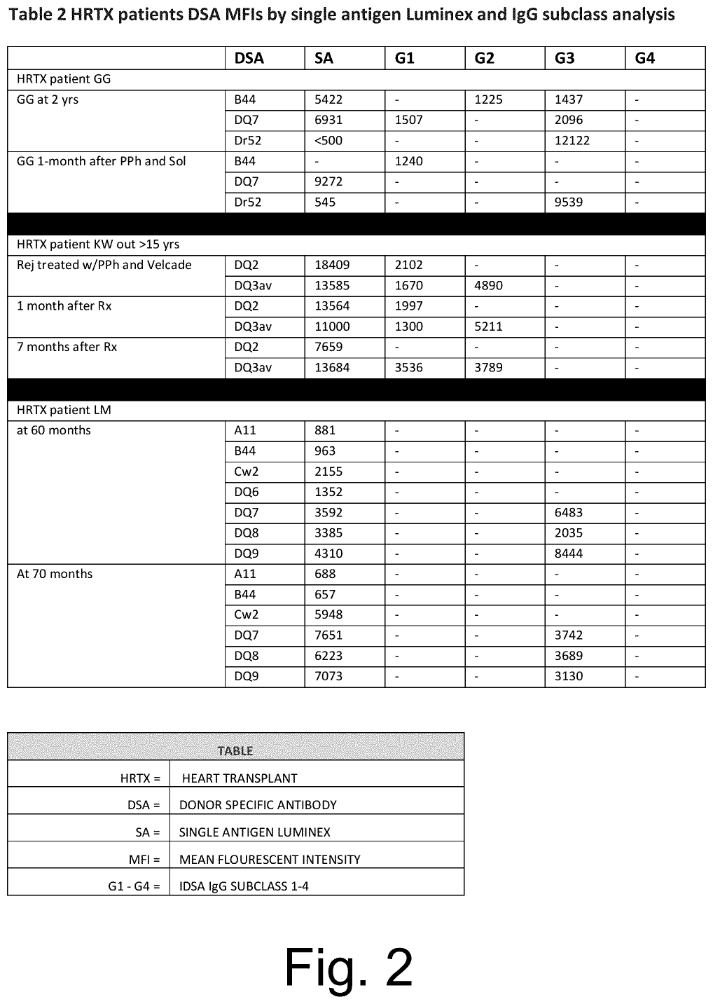

FIG. 3 is a table (Table 3);

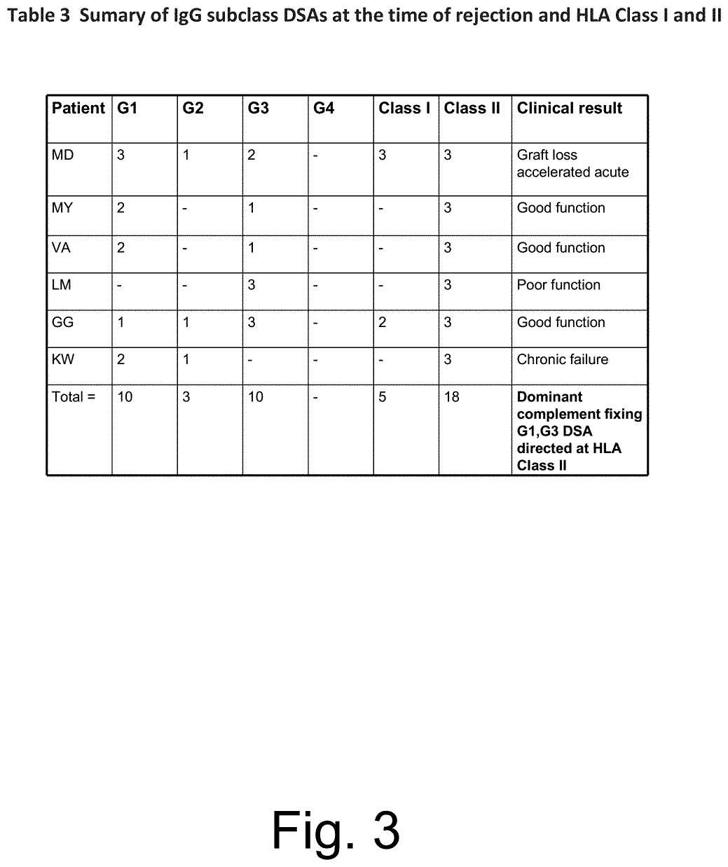

FIG. 4 is a table (Table 4);

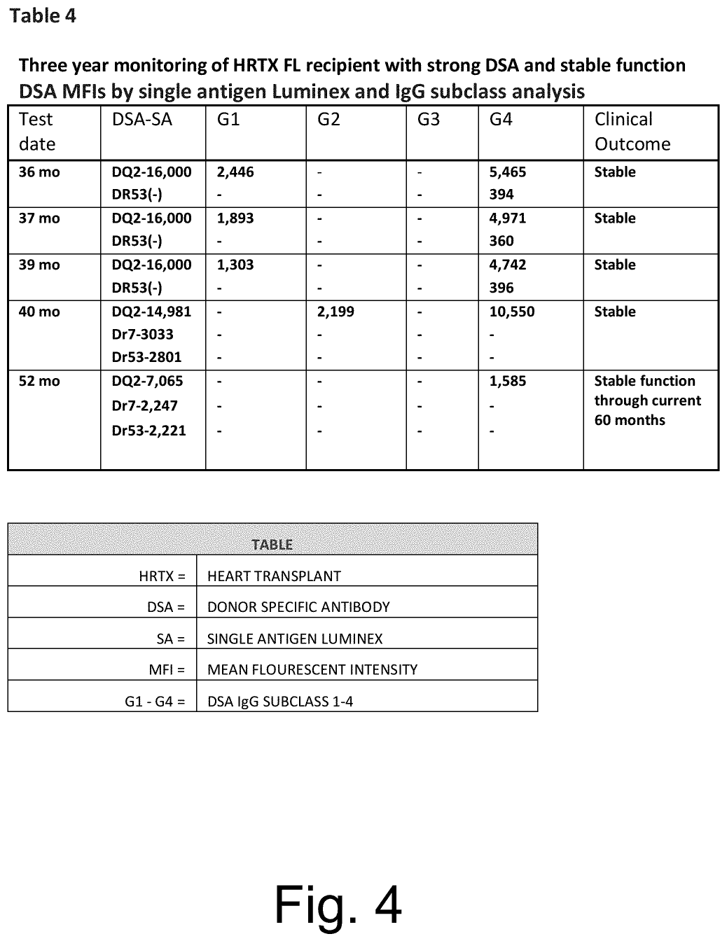

FIG. 5 is diagram of the immune system, as an example, with respect to a microbe as a source of antigens (e.g., soluble or cell surface antigens);

FIG. 6 is a diagraph of an example of basic structures of a immunoglobulin molecule;

FIG. 7 is a diagram of examples of structures for human IgG subclasses;

FIG. 8 is a table that shows some examples of information associated with IgG subclasses;

FIG. 9 is a table as to some examples of assays; and

FIG. 10 is a diagram as to some examples of assays.

### DETAILED DESCRIPTION

In placental mammals, antibody isotypes include immunoglobulin classes IgA, IgD, IgE, IgG and IgM, which differ in various aspects of their biological properties, functional locations, etc. As to the IgG class, it includes subclasses denoted G1, G2, G3, and G4, the numbers having been assigned based on data indicative of levels in serum for populations of individuals without immunological disorders, impairment, etc. (e.g., G1>G2>G3>G4).

As to some differences among the IgG subclasses, consider their ability to bind to various types of proteins found on certain cells involved in immunological activity. As an example, consider a protein known as an Fc receptor (FcR) that has an affinity for binding IgG subclasses G1 and G2 that exceeds its affinity for binding IgG subclasses G3 and G4. Indeed, for IgG subclass G4, such an FcR may exhibit little to no binding affinity. In general, it is possible to summarize some differences for IgG subclasses as follows: G1 and G3 have the property of complement fixation and binding to FcRs whereas G2 and G4 have poor or non-existent complement fixation (G4) and do not bind high affinity FcRs.

For transplant recipients, various donor-specific human leukocyte antigen (HLA) antibodies have been associated with rejection. HLA antibodies are typically not naturally occurring, with few exceptions they may be, for example, formed as a result of an immunologic challenge of a foreign material containing non-self HLAs via blood transfusion, pregnancy (paternally-inherited antigens), organ or tissue transplant, etc. Donor-specific HLA antibodies can include antibody isotypes of the class IgG. Thus, as an example, a transplant recipient may be tested for levels of one or more of the IgG subclasses G1, G2, G3 and G4. Such testing may indicate a level or levels of donor-specific HLA IgG subclass antibodies. As an example, a method may include testing levels of one or more of the IgG subclasses, for example, optionally using a testing kit or kits (e.g., and appropriate assay or assays).

As described herein, levels of donor-specific HLA IgG (DSAs) can play a role in transplant recipients. For example, data indicate that such DSAs play a role in heart transplant recipients (HRTX) and renal transplant recipients (RTR). In particular, data indicate that level of IgG subclass G4 plays a role, for example, where a higher level of G4 is beneficial to transplant recipients.

Data collected for a group of patients demonstrates that patients experiencing rejection had equal numbers of DSAs that were G1 and G3 but without DSAs that were G4. In the patients experiencing rejection, the predominant DSAs were directed to HLA Class II proteins. Also noted, even in patients subjected to successful rejection therapy, DSAs persisted, albeit in several instances exhibiting lowered mean fluorescent intensity (MFI) screening values (e.g., LUMINEX® screening technology, Luminex Corporation, Austin, Tex.). As an example, one HRTX recipient (HRTX1) was followed for a period of over 30 months. HRTX1 exhibited consistent continuous levels of DSAs that were G4 as well as stable cardiac function.

Class (e.g., or subclass) switch to G4 has been found to be controlled by T regulatory type 1 cells (Tr1) and interleukin 10 (IL-10), noting that Tr1 has been shown to be involved in production of immunosuppressive cytokines IL-10 and transforming growth factor beta (TGF-p). As to the beneficial nature of G4, it functions as a blocking antibody that does not bind to an FcR, inhibits the crosslinking of complement fixing G1, G3 and is a potential indicator of Tr1 tolerance induction.

### Example: G4 as a Blocking Antibody

Human immunoglobulin class IgG includes a G4 subclass that acts as a blocking antibody. Presence of G4 appears to be beneficial for transplant recipients; whereas, G1 and G3 appear to be detrimental for transplant recipients (e.g., as antibodies against HLA class I or II molecules).

### Example: Desensitization

As an example, a method can include utilization of HLA antigen (e.g., in the form of cells or purified HLA) administered (e.g., pre-transplant and/or post-transplant) using allergic desensitization protocols to induce a G4 tolerance response (e.g., increase levels of DSAs that are G4).

### Example: Increasing Levels of G4

G4 can be increased via various approaches. As an example, an indirect approach may be akin to desensitization where an agent/antigen is administered to a patient to promote G4 antibody production. As an example, a direct approach may include administration of G4. As another example, an approach is to modify cells to produce G4 and administer those cells. Such cells may be tissue (e.g., graft) cells. Approaches may differ temporally as to duration, time of application, etc. Approaches may consider conversion of IgG over time (isotype switch).

### Examples: Indirect, Direct and Other

As an example, an indirect approach can include administration of an agent that promotes G4 blocking antibody production. As an example, a direct approach can include injection of G4 blocking antibody. As an example, an approach can include modification of tissue cells to produce G4.

### Example: Types of Patients

As an example, a patient population may be selected for treatment based on one or more factors. As an example, one factor may be number of pregnancies (e.g., women having had multiple pregnancies being exposed to more “foreign” molecules). As an example, one factor may be exposure to an infective agent or agents.

### Example Indirect

As an example, an indirect approach may include determining sensitivities of patient (possible statistical analysis); administering proteins (e.g., HLA molecules) to desensitize (increase level of G4); monitoring patient IgG levels, including G4 blocking antibody level; and transplanting tissue (e.g., after a number of treatments, an achieved G4 level, a period of time, etc.).

### Example Direct

As an example, a direct approach may include determining whether a patient has low level of IgG4 blocking antibody or sensitivities; administering G4 blocking antibody (pre- and/or post-transplant); and transplanting or monitoring already transplanted patient.

### Example Other A

As an example, an approach may include treating tissue (e.g., genetically); testing tissue to see if producing G4 blocking antibody; and transplanting tissue.

### Example Other B

As an example, an approach may include transplanting tissue; transforming tissue to produce G4 blocking antibody; and monitoring patient (e.g., as to one or more levels of IgG subclasses, as to condition, etc.).

### Example: Assay

As an example, an assay may include one or more media that include molecules for binding one or more IgG subclasses. Such media may include beads, trays, etc. As an example, such media may include molecules with specificity to one or more proteins. For example, a molecule may include a structure of a HLA molecule (e.g., a donor-specific HLA molecule).

### Example: Bank of Proteins and Treatment

As an example, a bank of proteins may be provided. Testing of a patient may occur followed by selecting one or more of the proteins from the bank of proteins. Such testing may include using an assay.

As an example, a method may include testing a sensitized pre-transplant recipient candidate, determining one or more specific sensitivities of that candidate, and selecting one or more proteins from a bank of proteins. Such a method may optionally include modifying the one or more proteins. As an example, a mixture may be made of various proteins based at least in part on the aforementioned testing and determining. As an example, the proteins may include G4. For example, the proteins may include donor-specific HLA and/or donor-specific HLA G4.

As to treatment, a patient may be placed on a schedule to visit a clinic one a week or on another basis for purposes of receiving an injection or injections of proteins. Such a process may include monitoring, for example, for an end point as to level, ratio, etc., of G4 in the patient's blood.

As an example, a method may proceed without knowledge of a donor (e.g., donor tissue). For example, such a method may proceed based on knowledge of a patient's own sensitivities (e.g., per testing and determining).

As an example, a statistical analysis may be performed based on knowledge about a patient. For example, a patient may be given a questionnaire to determine factors that may help determine sensitivities. Further, information about likely donors may be taken into account (e.g., based on geography). For example, in Los Angeles, a donor may have a higher likelihood of being from one definable group or groups rather than another group based on population of those groups. Such an approach may be applied to various types of organs sought to be transplanted.

Example: Supplement with G4 Character

As an example, a method may include modification of a molecule with a G4 heavy chain. As an example, a recombinant G4 (e.g., isotype antibody) may be formed, for example, directed to a particular HLA. As an example, a molecule may be humanized (e.g., murine antibodies engineered as chimeric or humanized forms). As to regulatory approval, consider that since 1986 the US FDA has approved various antibody-based therapeutics for autoimmune indications as well as for cancer indications (e.g., including G4 antibodies). As an example, a pan-HLA antibody may be provided with G4 character.

### Example: Plasmapheresis

As an example, a method can include plasmapheresis, for example, to remove certain proteins from blood. Such a method may act to remove pre-existing donor-specific antibodies that could impact tissue to be received by a recipient. Such an approach may be combined with administration of a blocking antibody (e.g., G4).

Duration of G4 (e.g., direct, indirect or other)

As an example, a method may include administering blocking antibodies (e.g., G4) periodically (e.g., to a patient, to tissue, to tissue being cultured, etc.) where a half-life or clearance rate may be measured and, for example, used for future administration (e.g., to a patient, to tissue, to tissue being cultured, etc.). As an example, for a desensitization method, a different temporal mechanism may come into play. For example, desensitization can involve production of blocking antibodies (e.g., G4) as well as production of T cells that are tolerant (e.g., as in an allergic desensitization). As an example, a method may include a combination of therapies. For example, a desensitization method may be applied to a patient followed by administration of a blocking antibody. In such an example, desensitization may occur pre-transplant (e.g., until a level, ratio, etc., of G4 is reached) and administration may occur proximate to the time of transplant (e.g., to supplement desensitization).

As an example, post-transplant, a patient exhibiting chronic rejection may be experiencing conditions associated with HLA class II. In such an example, a desensitization process may be applied for HLA class II to raise the level of blocking DSAs (e.g., G4) in the patient.

### Pre-Transplant and Post-Transplant

As an example, a treatment may depend on time of transplant. As an example, a blocking antibody may be administered directly to a patient post-transplant. Such an approach may be beneficial compared to an approach that introduces an antigen to the patient where that antigen is to increase level of the blocking antibody (e.g., as the post-transplant patient may be under immunological stress).

### Various Allergy Studies

In bee or wasp venom immunotherapy, G4 has been considered where IL-10 makes B cells switch the produced immunoglobulin class from IgE to IgG subclass 4 (G4) (see, e.g., Punnonen et al., “Interleukin 13 induces interleukin 4-independent IgG4 and IgE synthesis and CD23 expression by human B cells”, Proc Natl Acad Sci USA. 1993 Apr. 15; 90(8): 3730-3734, which is incorporated herein by reference).

A study by Savilahti et al. (“Early recovery from cow's milk allergy is associated with decreasing IgE and increasing IgG4 binding to cow's milk epitopes”, J Allergy Clin Immunol. 2010 June; 125(6): 1315-1321.e9, which is incorporated herein by reference) stated: “Attaining tolerance to cow's milk is associated with decreased epitope binding by IgE and a concurrent increase in corresponding epitope binding by IgG4.”

A study by Cady et al. (“IgG antibodies produced during subcutaneous allergen immunotherapy mediate inhibition of basophil activation via a mechanism involving both FcγRIIA and FcγRIIB”, Immunol Lett. 2010 May 4; 130(1-2): 57-65, which is incorporated herein by reference) stated: “Effects of IT may also be mediated by allergen specific IgG antibodies”; “Successful immunotherapy is usually correlated with an initial increase in allergen-specific IgG1, followed by an increase in IgG4 and, much later, a decrease in allergen-specific IgE antibodies”; “Allergen-specific IgG4 may act to block the interaction of IgE with allergen, decreasing CD23-mediated B cell allergen uptake and subsequent antigen processing and decreasing IgE mediated degranulation of mast cells and basophils”; and “Modest elevations of IgG1 and IgG2, and especially IgG4 Abs were associated with cat keeping”. As an example, a method may involve use of a mechanism that includes cross-linking for complement activation and, for example, a mechanism that also interferes with the binding of an allergen to IgE.

### Example: Autoimmune Disease

As an example, a therapy may be applied to a patient experiencing an autoimmune disease. For example, a direct and/or indirect method for treatment may prove useful in autoimmune disease particularly where the target is known (e.g., in MS, IgG4 specific to myelin basic protein).

As an example, a direct method may be implemented that supplies specific G4. As an example, an indirect method may be implemented that uses antigens to generate G4 and tolerance.

As an example, donor specific antibody DSA refers to antibody directed to HLA which constitute, for example, most of the major antigens that are responded to in solid organ and stem cell transplants and therefore are responsible for graft failure and GvHD in stem cell TX.

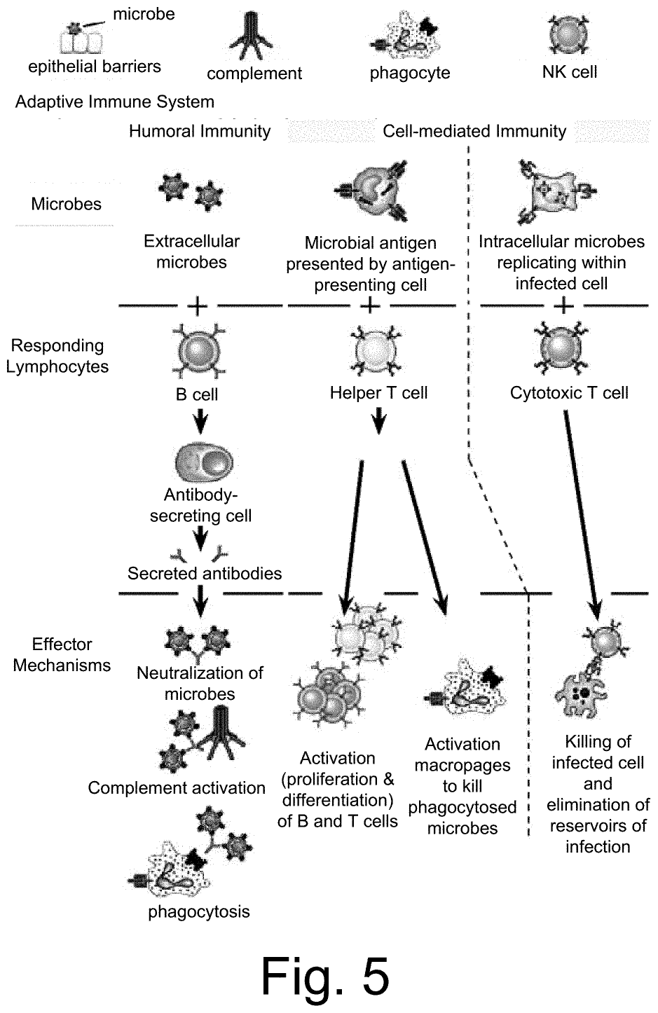

FIG. 5 shows a diagram of the immune system, as an example, with respect to a microbe as a source of antigens (e.g., soluble or cell surface antigens); noting that other sources of antigens may be explained to varying extent via the diagram. For example, FIG. 5 shows that in humoral immunity, B cells recognize soluble or cell surface antigens (on extracellular microbes) and differentiate into antibody-secreting plasma cells. In cell-mediated immunity, helper T cells recognize antigens on the surfaces of antigen-presenting cells and secrete cytokines, which stimulate B cells and T cells; cytotoxic T cells recognize antigens on the infected cells and kill these cells.

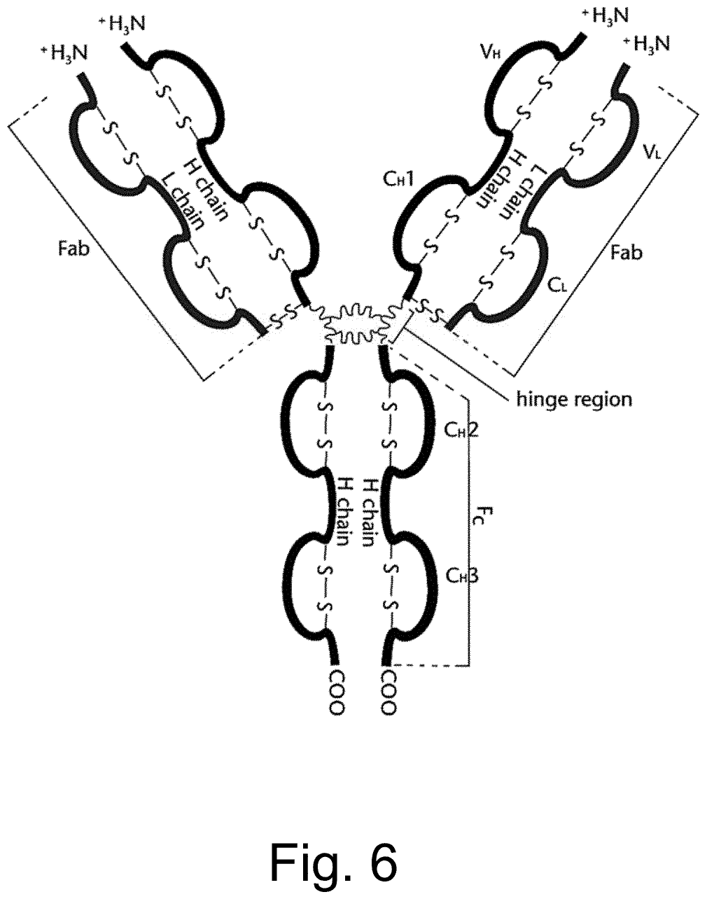

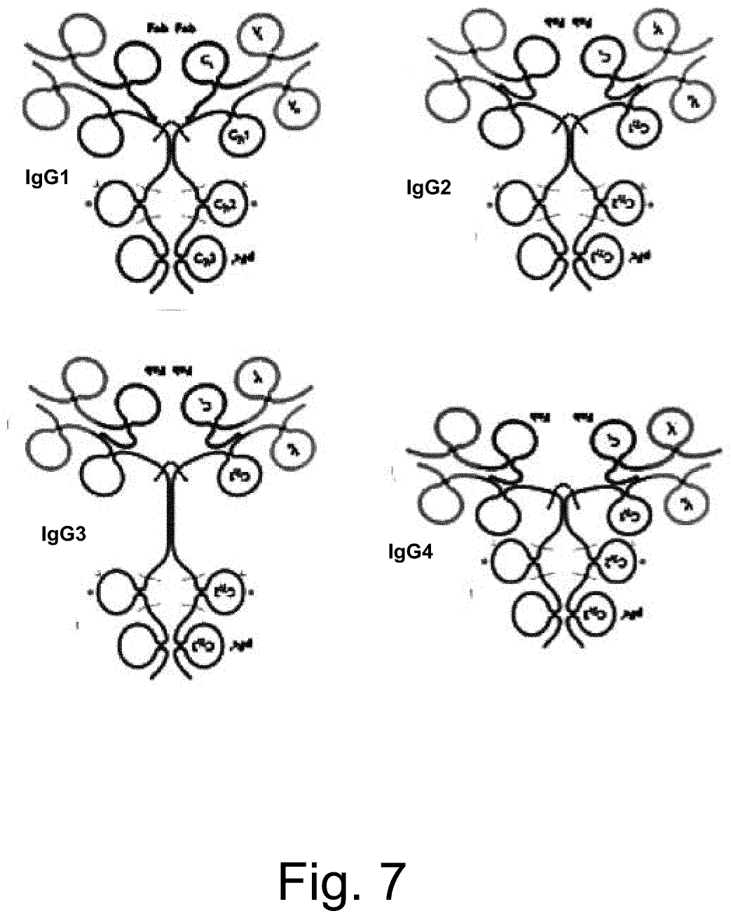

FIG. 6 shows a diagraph of an example of basic structures of an immunoglobulin molecule and FIG. 7 shows a diagram of examples of structures for human IgG subclasses. For example, FIG. 6 shows a schematic drawing of the basic structure of the human immunoglobulin molecule. The amino-terminal end is characterized by sequence variability (V) in both the heavy and light chains, referred to as the VH and VL regions respectively. The rest of the molecule has a relatively constant (C) structure. The constant portion of the light chain is termed the CL region or domain. The constant portion of the heavy chain is further divided into three structurally discrete regions: CH1, CH2 and CH3. The hinge region is a segment of the heavy chain, located between the CH1 and CH2 domains. Carbohydrate groups are attached to the CH2 domains of the heavy chains. In FIG. 6, Fab is Fragment antigen binding; and Fc is Fragment crystallizable. As to FIG. 7, for example, it shows schematic structure of the four human IgG subclasses. The four domains of the heavy chains and the two domains of the light chains are shown. The disulfide bonds are shown as are each site of attachment of a carbohydrate side chain as well as the sites involved in the activation of complement and the C1q binding sites. The major cleavage points exist for pepsin and papain. After cleavage with pepsin or papain, different fragments may be generated.

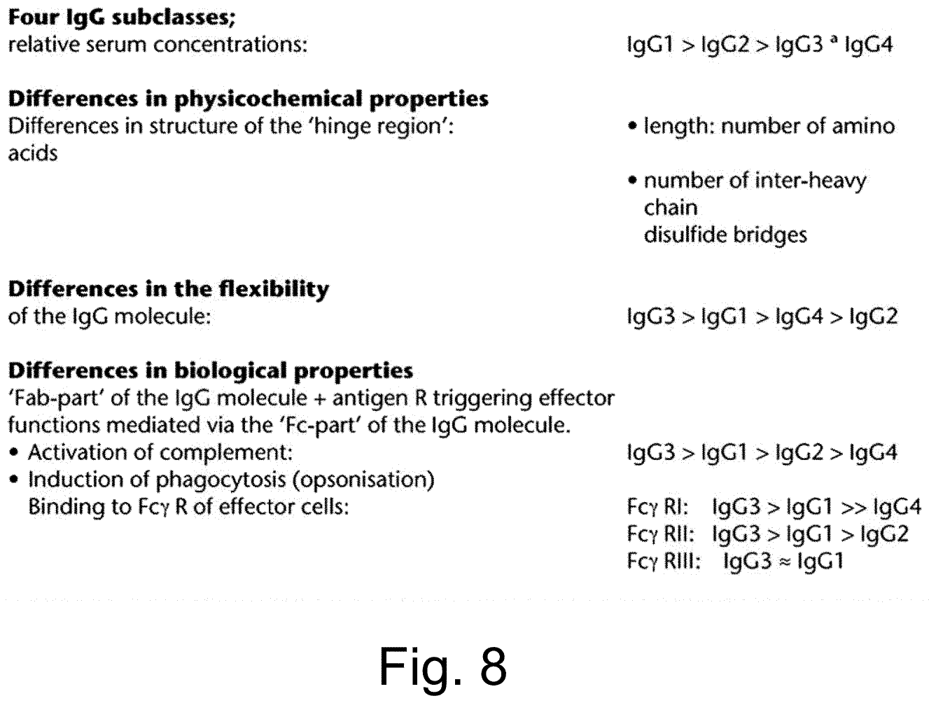

FIG. 8 shows a table that shows some examples of information associated with IgG subclasses. For example, some information is shown as to properties, flexibility, etc.

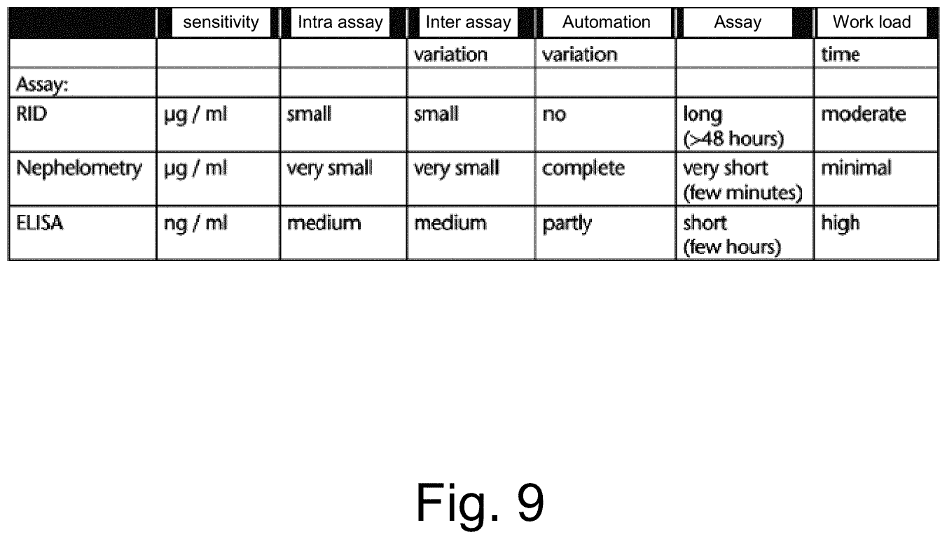

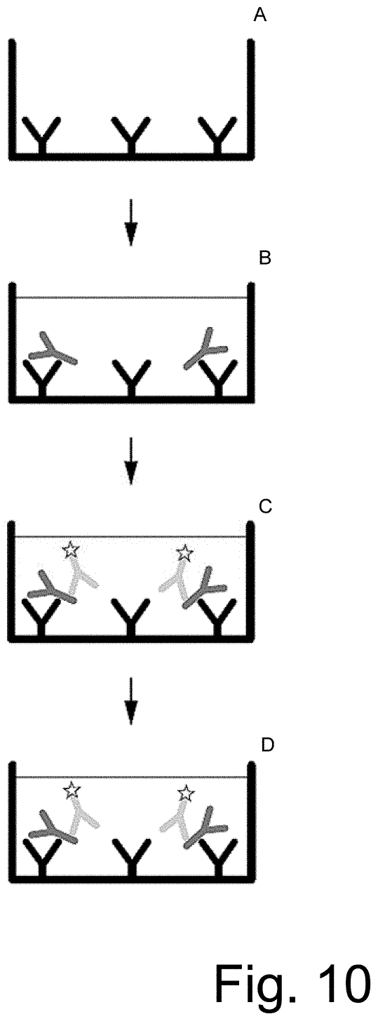

FIGS. 9 and 10 show a table and a diagram as to some examples of assays. As to the diagram, it pertains to an IgG subclass ELISA. While the examples of FIGS. 9 and 10 show monoclonal anti-human IgG subclass-specific antibody coated on a well of a microtitre plate, one or more other sources of anti-IgG sublass-specific antibodies may be provided. As an example, a method may implement one or more of the assays shown in the table of FIG. 9 (e.g., RID, Nephelometry, ELSIA).

As an example, a machine may include one or more processors and a memory device with memory accessible by at least one of the one or more processors. As an example, a method may be implemented, for example, at least in part, in the form of instructions stored in one or more computer-readable storage media (e.g., processor-readable storage media) where the instructions are computer-executable (e.g., processor-executable), for example, to instruct a machine to perform one or more actions. As an example, a computer-readable storage medium may be a storage device (e.g., a non-transitory medium and not a carrier wave). Circuitry of a machine may include circuitry such as that of a machine for use in performing an assay (e.g., RID, Nephelometry, ELISA, etc.).

As an example, data may be stored in one or more storage devices, for example, as a database. As an example, one or more algorithms may be implemented for accessing, analyzing, etc., data in a database, whether associated with data in a single storage device or distributed storage devices (e.g., networked storage devices). As an example, one or more computers may include network interfaces, for example, to access one or more storage devices and/or other devices (e.g., computing devices, etc.). As an example, an algorithm may be a statistical and/or probabilistic algorithm, for example, that can output statistics, probabilities, etc. Such an approach may allow for analyzing data for a patient, selecting a particular treatment for a patient, etc. As an example, an algorithm or algorithms may be implemented, in part, via processor-executable instructions. For example, a computer may be configured to receive information, to process information and to output information (e.g., based in part on the received information and processing of information). For example, information about immunological status of a person or persons (e.g., or tissue or tissues) may be input and processed, optionally with respect to one or more databases of information, and information output. Output information may pertain to one or more aspects of a transplant tissue, tissues, donor, recipient, etc. As an example, information output may include matching information for a recipient and, for example, risk information, for example, based at least in part on one or more levels of an IgG (e.g., an IgG subclass or subclasses, etc.). As mentioned, ratios of IgG subclasses may be beneficial as to making an assessment, planning treatment, etc.

Some Examples from Trials

### Some Abbreviations Used in Description of Trials:

DSA=Donor Specific Antibody

RTR=Renal Transplant Recipient

SCr=Serum Creatinine

MFI=Mean Fluorescent Intensity

SA=Single Antigen

FcR=High affinity Fc receptor

IgG=Immunoglobulin G

TX=Transplant

FCXM=Flow Cytometry Cross match

G1, G2, G3, G4=IgG1, IgG2, IgG3, IgG4 subclasses

HLA DSA is a risk factor for graft survival and has been linked with rejection and poor prognosis (1,2). However, registry data analysis of patients with graft loss has showed that DSA is not significantly associated with graft failure where DSA was >1000 MFI (3), and some have considered preformed and/or de novo DSA low risk factors for transplants (4). Therefore the conjecture of HLA DSA hazards is an open question.

As an example, assessing HLA DSA risk may be achieved using one or more methods that include, for example, a solid phase assay or assays. As an example, total IgG may be measured in various tests used yet, as noted herein, the subclasses of IgG are relevant in immune responses (5,6). Data indicate that these IgG subclasses vary greatly in their ability to cause inflammation and tissue destruction. There are four subclasses of IgG1-4 (G1, G2, G3, and G4) that have very different properties for complement fixation and antibody carboxyl-terminal binding to Fc receptors. G1 and G3 activate complement and function through phagocyte high affinity Fc receptors. G2 is a poor complement activator and low binding to high affinity Fc-receptors. G4 has no complement fixing activity and binds to an inhibitory Fc receptor (7). Therefore, in transplant patients with Gland G3, DSAs are likely to have a high probability of graft destruction through complement activation and phagocytosis; whereas, for example, G2 and/or G4 may have a beneficial effect.

In seven case studies, various analyses were performed for subjects that included 3 renal and 4 heart transplant patients where, for example, analyses provide data for HLA DSA G1-G4 subclasses. Some results from these analyses are described herein, for example, with some discussion as to DSA IgG subclass analysis and its relevance in the rejection, graft survival outcome potential manipulation of IgG subclass switching.

### Material and Methods

### HLA Typing

HLA Class I & II antigens were detected using the standard monoclonal trays using the micro-cytotoxicity method (One Lambda, Inc., Canoga Park, Calif.). In the case of ambiguous results, or uncertainty; additional testing using PCR-SSP primers were obtained and used (One Lambda, Inc., Canoga Park, Calif.) and PCR-SSP (QIAGEN Inc., Valencia, Calif.).

### HLA Single Antigen Specificity Analysis (SA)

All RTR were tested for the presence of antibodies of IgG isotype against HLA-A, -B, -DR (1-18), -DRw (51, 52, and 53), -DQ (1-9), using SAB Luminex technology (Lab Screen Single Antigen, One Lambda, Inc., Canoga Park, Calif.). The tests were performed according to the vendor's instructions. Antibody specificity was analyzed manually using baseline MFI values. Positive MFI thresholds were defined on the basis of >500 MFI SA when a DSA was noted for a donor mismatched HLA antigen.

### IgG Subclass Determination

As an example, SA Luminex technology was used to determine the specificity of HLA class I & II IgG antibody subclasses. As an example, a test was modified into a “sandwich assay” where graft recipient serum was reacted with SA Luminex beads, followed by murine IgG subclass specific monoclonal antibodies, and then a polyclonal PE conjugate (8). Briefly, the human IgG-specific secondary antibodies conjugated to PE were replaced with murine monoclonal antibodies specific for human G1, G2, G3, and G4 subclasses and a goat F(ab)2 polyclonal antibody anti murine PE conjugate. Negative serum (containing no HLA antibodies) had no reactivity with murine monoclonal specific to G1-G4 subclasses. Pooled positive anti HLA sera (positive control) had reactivity to G1-G4 subclass specificities, specific beads had patterns of reactivity were inclusive of several subclasses whereas other beads were specific to only one subclass and specificity. Replicate samples and controls had MFI's and CV's within 5%. Positive controls consistently showed the same pattern of bead and subclass reactivity.

### Rejection

As an example, RTR rejection was determined by a rise of 0.1 mg/dl/day for consecutive days, DSA and/or biopsy. Hrtx recipients showed left ventricular (LV) dysfunction as exhibited by symptom of decreased ejection fraction (EF) and/or biopsy.

### Flow Cytometry Cross Match (FCXM)

Some details of the procedure for three color flow cross match with pronase have been described elsewhere (9). Bortezomib was used according to the protocols described by Everly et al (10).

### Results:

G1-G4 subclass DSA analysis provides a useful tool, for example, as compared to the SA Luminex. MFI in the SA Luminex are not directly comparable since the PE developing antibodies and the murine subclass specific antibodies are not part of the SA Luminex. In case study analysis, there were instances when the DSA subclasses (MFI>500) are found when the SA Luminex was below were below a selected “standard” measurable range of 500 MFI. As an example, the mechanism may be a function of a small amount of high affinity DSA that is magnified by binding of the monoclonal antibodies and goat F(ab)2 PE antibodies used in the sandwich subclass G1-G4 assay. Likewise, in the SA Luminex, DSA was present when no G1-G4 subclasses were seen. This may be caused by low affinity of the DSA which is washed off in the multiple steps in the subclass assay.

Table 1 shows the DSA results for renal TX recipient MD, a 40 year old single parous African American female who received a kidney from her husband. Prior to TX the patient had no antibodies demonstrated by SA Luminex however, there was a weak G1 B44 DSA demonstrated retrospectively by G1-G4 subclass analysis. The FCXM was negative with pronase but historical (greater than 6 months) sera were not available. MD presented with anuria and pain over graft on POD 5. At this time SA Luminex showed seven DSAs. The strongest was an A29 which was a G1 (5116 MFI), and along with the G1 DSA to B44, no other G1-G4 DSAs were found. The patient was treated with PPh (x5), Mg, and Bortezomib. By POD 9 SA Luminex DSA were represented predominately by G1 and G3 and relatively strong as compared to the SA Luminex. Also on POD 9 HLA-B63 was not found in the subclass analysis, but perhaps notably SA Luminex DSAs, which were weak, were much stronger in the subclass analysis (i.e. MFI for A29 1,232 SA versus 11,768 for G1 MFI on POD9). Furthermore, Dr7 and DR53 were not found in the SA DSA but showed up as G1and/or G3 subclass DSA. On POD 30 there were still strong complement fixing DSAs and the patient had lost function and was nephrectomized on POD 33. The persistence of the antibody throughout the transplant course and anti-rejection treatment may be, for example, a function of the complement fixing DSA, anamnestic response, memory cells, and high responder status for MD. Five months after nephrectomy, G1 and G3 persisted in all the SA DSAs, but there was a subclass to G4 subclass in A33 and Dr7.

MY (Table 1) was negative for T and B cell FCXM, but presented with rejection 3 weeks after TX. Pre TX serum was found to have a DSA to Dr4 SA (weak 1330 MFI) and G3 DSA to Dr4, (1232 MFI) (table 1). At the time of rejection DSA to HLA-B18 was not found in the subclass analysis, but DSA to Dr4 was G1, G3 and Dr53 was G1. MY had DSAs, rising sCr, and a fractured kidney. The graft was surgically repaired and treated with Bortezomib PPh X3 and Mg. A weak DSA persists 18 months later with the current sCr of 1.0.

VA (table 1) was a positive pronase B-cell FCXM with a 156 MCS, but negative T-cell FCXM with DSAs B8, B39, DQ7, and Dr52. Pre-TX the recipient had a Dr52 DSA SA (3869 MFI) which was G3 (6091 MFI). Pre-and perioperative to TX, VA was treated with Bortezomib, PPH x3, Mg, and Rituxan. A rising sCr diagnosed as rejection occurred five months post-TX. Three DSAs were found by SA which showed G1 and G3 by subclass analysis (table 1B). VA was treated with anti-rejection therapy and sCr was lowered. At seven months post-TX VA had only a weak (1,367 MFI) Dr52 DSA by SA, but subclass analysis showed a strong G3 DSA to Dr52, at 12,071 MFI. With follow-up at 24 months, VA still shows the Dr52 (G3; 763 MFI), but no other DSAs and has a sCr of 0.9mg/dl. VA is an example of no apparent graft dysfunction even though there is a chronic G3 DSA.

In both MY and VA rejection was resolved and sCr is normal. However, as an example, it may be arguable that subclinical chronic rejection is occurring. In IgG subclass testing, these two patients have a have G1 and/or G3 subclass DSA that are lowered as compared to the time of rejection or positive FCXM, but still represent risk to outcome.

HRTX GG (Table 2) was admitted at 2 years with an EF of 33%. SA Luminex showed a B44, DQ7and Dr52 DSA which had strong G3 subclass components. Patient was treated with PPh X3 and solumedrol. One month after treatment GG had persistent DQ7 by SA Luminex, but only B44 and Dr52 by subclass analysis. Currently, the DQ7 persists by SA and EF is normal.

HRTX KW is presented with LV dysfunction EF 38% and DSA by SA Luminex to DQ2, DQ3, which were G1 and G2. Patient was treated with Bortezomib and PPh after one month DSA SA to DQ2 and DQ3 and G1, G2 with continued. The EF increased to 53% and DQ3 G1 and G2 were found, but no corresponding subclass to DQ2.

HRTX LM was transplanted in 2003 and had multiple rejection episodes. During rejection at 60 months the patient was treated with PPh x3 and lvlg. G3 antibodies continued when tested at 70 months. LM is currently doing poorly and has been relisted.

To summarize, in the cases presented as examples herein, and at the time of rejection (Table 3), there were 10 G1, 10 G3, and 3 G2 subclasses and of these 23 IgG subclass DSAs, 18 were HLA Class II antibodies. G3 and G1 are sequentially the first two IgG subclasses to be produced by gene deletion. G3 and G1 both activate complement and bind to phagocyte's Fc receptors. These DSAs were found concomitant to graft rejection. Class II HLA DSA was found potentially caused by up-regulated Class II expression during rejection.

The data for FL is noteworthy. Transplanted in 2006 FL was found, through routine DSA monitoring, to have a strong DSA by SA luminex greater than 16,000 MFI (Table 4). The HRTX recipient had no LV dysfunction and was analyzed for IgG subclass and found to have a G1, strong G4 and transient G2. The graft function and DSA subclass has been stable for the last three years. G4 is the typical blocking antibody which appears in chronic stimulation and is regulated through Th1 (11,12).

Discussion of Some of the Data/Studies/Analyses

HLA DSA may be a probable risk factor for transplant recipients (1,2,13,14) however, the extent of the risk has to be measured in the quantity, number, complement fixing, and FcR binding of the antibodies. In the case studies presented there was preponderance of complement fixing DSA at the time of rejection, and some which continued even after the rejection ameliorated. As an example, persistent HLA DSA may eventually result in graft rejection (15). However, in patients that are doing well with 18-24 month follow up (GG, MY and VA) DSA was lowered or disappeared by subclass analysis after rejection treatment. Therefore, as an example, IgG subclass analysis may be helpful in order to determine risk. As an example, complement fixing Cl q binding and G3 DSA has been shown to be a risk factor for renal and liver transplant recipients (16,17). In case studies described herein, as an example, G1 and G3 were found to be associated with rejection and more class II DSA was directed against HLA DQ after one year post transplant.

As an example, the FL case shows a recipient with stable function and a G4 DSA which is non complement fixing and non binding to high affinity FcR (7) i.e. a blocking antibody. FL was monitored for over two years with most recent subclass analysis showing only G4. There may be many cases like FL which are not discovered since these patients are stable with no dysfunction and therefore not analyzed for DSA. For example, a recent report exists of a pre transplant G4 DSA positive crossmatch that was successfully transplanted (18). G4 is a blocking antibody that may be relatively common in patients with DSA and successful transplants. Hence a discussion of G4 occurrence is particularly apropos to a tolerant state and stable function in solid organ transplants. Some knowledge and understanding of specific G4 comes by way of allergic desensitization where allergens are given at increasing doses in 2-4 week intervals (19). Regulatory T cell (Treg), particularly Tr1, are generated initially (20,21) and increase in frequency in 3-6 months. In allergic desensitization, Tregs produce high levels of anti inflammatory cytokines IL-10 and TGF-beta (21,22). Tr1 controls the Ig class switch to G4 and increased production of IL-10 controls and high affinity G4 production (20). For example, high affinity DSA subclasses are predicted in the assay technique herein and high affinity G4 may be the most effective in eliminating allergic symptoms. Allergen specific G4 is increased 10-100 fold in successful desensitization (23) and is used as a measure for desensitization. Not only is G4 non complement activating and non-FcR binding, but it inhibits immune complex formation and can inhibit crosslinking and complement of Gland G3 Ig subclasses (20). Therefore, G4 has anti-inflammatory properties and inhibition of crosslinking explains the salutary effect even in the presence of G1 and G3, as was seen in some of the early samples tested from HRTX FL.

As an example, appearance of G4 in solid organ transplants may act as a protective and a potential indicator of the tolerance induction and Tr1 Tregs. As with allergic desensitization, the HLA antigen, for example, in the form of cells or purified HLA, may be administered pretransplant or post transplant (or both) using an allergic desensitization model and protocol in order to induce tolerance and G4 blocking antibody.

### References (Incorporated by Reference Herein):

[1] Terasaki P I, Ozawa M. Predicting kidney graft failure by HLA antibodies: a prospective trial. Am J Transplant 2004; 4:438.

[2] Arnold M L, Pei R, Spriewald B, Wassmuth R. Anti-HLA class II antibodies in kidney retransplant patients. Tissue Antigens 2005; 65:370.

[3] Susal C, Ovens J, Mahmoud K, Dohler B, Sherer S, Rhuenstroth A, et al. No association of kidney graft loss with donor specific human leukocyte antigen antibodies. Transplantation 2011;91(8):883.

[4] Guidelines for the detection and characterisation of clinically relevant antibodies in transplantation. Available at: www.bshi.org.uk/BTS/BSHI. Accessed Oct. 12, 2010

[5] Aalberse R C, Schuurman J. IgG4 breaking the rules. Immunology 2002;105:9-19.

[6] Van der Neut Kolfschoten M, Schuurman J, Losen M, Bleeker W K, Martinez-Martinez P, Vermeulen E et al. Anti-inflammatory activity of human IgG4 antibodies by dynamic Fab arm exchange. Science 2007;317:1554-1557.

[7] Van der Neut Kolfschoten M, Schuurman J, Losen M, Bleeker W K, Martinez-Martinez P, Vermeulen E et al. Anti-inflammatory activity of human IgG4 antibodies by dynamic Fab arm exchange. Science 2007;317:1554-1557

[8] Cicciarelli J, Kasahara N, Pitpitan, Houston k, Hutchinson I, and Shah T. IgG subclass antibodies in transplant recipient sera: An IgG subclass specific assay to determine the “good” and “bad” IgG antibodies. Visuals of the Clinical Histocompatibility Workshop 2009 pp 42-43. P I Terasaki ed. One Lambda Inc, 21001 Kitridge St, Canoga Park Calif. 91303.

[9] Vaidya S, Cooper T Y, Avandsalehi J. Improved flow cytometric detection of HLA alloantibodies using pronase: potential implications in renal transplantation. Transplantation 2001; 71(3):422-8.

[10] Everly M J, Everly J J, Susskind B, Brailey P, Arend L J, Alloway R R, et al. Bortezomib provides effective therapy for antibody- and cell-mediated acute rejection. Transplantation: 2008(86): pp 1754-1761.

[11]Akdis M, Blaser K, Akdis C A. T regulatory cells in allergy: novel concepts in the pathogenesis, prevention, and treatment of allergic diseases. J Allergy Clin Immunol 2005;116:961-968; 969.

[12] Akdis C A, Akdis M, Blesken T, Wymann D, Alkan S S, Muller U et al. Epitope-specific T cell tolerance to phospholipase A2 in bee venom immunotherapy and recovery by IL-2 and IL-15 in vitro. J Clin Invest 1996;98:1676-1683.

[13] Vincent A, Venetz J-P, Pantaleo G, Pascual M. Low levels of human leukocyte antigen donor-specific antibodies detected by solid phase assay before transplantation are frequently clinically irrelevant. Hum Immunol 2009;70(8):580-3.

[14] Heinemann F M, Roth I, Rebmann V, Arnold M L, Witzke O, Wilde B, et al. Immunoglobulin isotype-specific characterization of anti-human leukocyte antigen antibodies eluted from explanted renal allografts. Hum Immunol 2007;68(6):500-6.

[15] Lachmann, Nils; Terasaki, Paul I.; Budde, Klemens; Liefeldt, Lutz; Kahl, Andreas; Reinke, Petra; Pratschke, Johann; Rudolph, Birgit; Schmidt, Dank); Salama, Abdulgabar; Schönemann, Constanze. Anti-Human Leukocyte Antigen and Donor-Specific Antibodies Detected by Luminex Posttransplant Serve as Biomarkers for Chronic Rejection of Renal Allografts. 2009—Volume 87—Issue 10—pp 1505-1513

[16] Hugo Kaneku, J O'Leary, M Taniguchi4, B. Susskind3, P. I. Terasaki, G B. Klintmalm. Donor-specific human leukocyte antigen antibodies of the immunoglobulin G3 subclass are associated with Chronic rejection and graft loss after liver transplantation. 2012; 18(8): 984-992.

[17] Sutherland S M, Chen G, Sequeira F A, Lou C D, Alexander S R, Tyan D B. Complement-fixing donor-specific antibodies identified by a novel C1q assay are associated with allograft loss. Pediatr Transplant. 2012; 16(1):12-7

[18] A. Lobashevsky, K. Rosner, W. Goggins, N. Higgins Subtypes of immunoglobulin (Ig)-G antibodies against donor class II HLA and cross-match results in three kidney transplant candidates. Transplant Immunology 23 (2010) 81-85

[19] L Cox, M Calderon, amd O Pfaar. Subcutaneous allergen immunotherapy for allergic disease. Immunotherapy 2012. 4:601-616.

[20] M. Jutell & C. A. Akdis Immunological mechanisms of allergen-specific Immunotherapy. Allergy 2011; 66: 725-732.

[21] Francis J N, Till S J, Durham S R. Induction of IL-10+ CD4+ CD25+ T cells by grass pollen immunotherapy. J Allergy Clin Immunol 2003;111:1255-1261

[22] Ling E M, Smith T, Nguyen X D, Pridgeon C, Dallman M, Arbery J et al. Relation of CD4+ CD25+ regulatory T-cell suppression of allergen-driven T-cell activation to atopic status and expression of allergic disease. Lancet 2004;363:608-615.

[23] Reisinger J, Horak F, Pauli G, van Hage M, Cromwell O, Konig F et al. Allergen-specific nasal IgG antibodies induced by vaccination with genetically modified allergens are associated with reduced nasal allergen sensitivity. J Allergy Clin Immunol 2005;116:347-354.

Although some examples of methods, devices, systems, arrangements, etc., have been illustrated in the accompanying Drawings and described in the foregoing Detailed Description, it will be understood that the example embodiments disclosed are not limiting, but are capable of numerous rearrangements, modifications and substitutions.

What is claimed is:

1. A method comprising:
determining a donor-specific human leukocyte antigen of a tissue transplant recipient;
determining a ratio of the tissue transplant recipient for a level of an immunoglobulin G subclass 4 antibody (G4) for the antigen with respect to a level of another immunoglobulin G subclass antibody for the antigen; and
based on the ratio, deciding to administer an amount of the G4 to the tissue transplant recipient.

2. The method of claim 1, comprising administering the amount of the G4 to the tissue transplant recipient.

3. The method of claim 1, further comprising making the G4.

4. The method of claim 1, further comprising determining another donor-specific human leukocyte antigen of the tissue transplant recipient.

5. The method of claim 1, wherein one or more non-binding portions of the G4 block an immune response cascade.

6. The method of claim 1, comprising monitoring level of the G4 in the recipient.

7. The method of claim 6, comprising repeating the decision to administer based at least in part on the monitoring.

8. The method of claim 7 comprising, prior to repeating the decision to administer, adjusting at least a G4 dose amount or a G4 dose frequency based on the ratio.

9. The method of claim 8, comprising administering the adjusted G4 dose amount.

10. The method of claim 8, comprising administering the G4 dose according to the adjusted frequency.

11. The method of claim 1, comprising comparing a monitored level of the G4 of the tissue transplant recipient to a target level of the G4 for the tissue transplant recipient.

12. A method comprising:
determining a donor-specific human leukocyte antigen of a prospective tissue transplant recipient;
administering the donor-specific human leukocyte antigen to the prospective tissue transplant recipient to increase level of an immunoglobulin G subclass 4 antibody (G4); and
monitoring level of the G4 in the prospective tissue transplant recipient.

13. The method of claim 12, comprising deciding to transplant the prospective tissue transplant recipient with tissue based at least in part on the monitoring.

14. The method of claim 13, comprising performing the transplant such that the prospective tissue transplant recipient is a tissue transplant recipient.

15. The method of claim 14, comprising administering the G4 antibody to the tissue transplant recipient.
